(19) United States
(12) Patent Application Publication
(10) Pub. No.: US 2025/0147137 A1
(43) Pub. Date: May 8, 2025

(54) MAGNETIC RESONANCE IMAGING APPARATUS AND CONTROL METHOD OF MAGNETIC RESONANCE IMAGING APPARATUS

(71) Applicant: FUJIFILM Corporation, Tokyo (JP)

(72) Inventor: Takashi NISHIHARA, Tokyo (JP)

(73) Assignee: FUJIFILM Corporation, Tokyo (JP)

(21) Appl. No.: 18/940,748

(22) Filed: Nov. 7, 2024

(30) Foreign Application Priority Data
Nov. 8, 2023 (JP) 2023-190620

(51) Int. Cl. G01R 33/56; A61B 5/026; A61B 5/055; G01R 33/54

(52) U.S. Cl. CPC G01R 33/5607; A61B 5/0263; A61B 5/055; G01R 33/546

(57) ABSTRACT

Provided is an MRI apparatus that can suppress fat, minimize an influence on T1 contrast or T2 contrast, and acquire an image in which has excellent discrimination ability between inflamed tissue and normal tissue.

In imaging using a fat suppression IR pulse, an MSDE sequence is added immediately before a data collection sequence to suppress a blood flow. Desired contrast is estimated based on imaging conditions and imaging parameters of the data collection sequence, and a duration of the MSDE sequence and a delay time of the data collection sequence started from the MSDE sequence are adjusted such that the desired contrast is obtained.

### CROSS-REFERENCE TO RELATED APPLICATIONS

The present application claims priority under 35 U.S.C. § 119 to Japanese Patent Application No. 2023-190620, filed Nov. 8, 2023. Each of the above application(s) is hereby expressly incorporated by reference, in its entirety, into the present application.

### BACKGROUND OF THE INVENTION

### 1. Field of the Invention

The present invention relates to a magnetic resonance imaging apparatus, and particularly relates to a fat suppression imaging technique.

### 2. Description of the Related Art

As one of imaging methods using a magnetic resonance imaging apparatus (hereinafter, referred to as an MRI apparatus), there is a method (fat suppression imaging) of suppressing a signal from fat and enhancing the ability of visualizing a tissue other than the fat. As the method of suppressing the signal from the fat, various methods have been proposed, such as a method of applying an RF pulse (referred to as a fat-sat pulse or a CHESS pulse) having a magnetic resonance frequency of the fat to saturate the signal from the fat and then executing a pulse sequence for data collection, and a method of applying an IR pulse to invert spins and using a difference in an inversion recovery rate between a fat tissue and other tissues to execute a pulse sequence for data collection at a point in time TI (null point) at which the signal from the fat is zero (STIR method) (Japanese Patent No. 6420583, JP2018-202122A, and the like).

Japanese Patent No. 6420583 discloses that, after TI, a data collection pulse sequence is executed twice after applying a fat suppression pulse, and low frequency region data and high frequency region data of k-space are collected. In the technique disclosed in Japanese Patent No. 6420583, the low frequency region data is collected at a timing at which a nuclear spin (also referred to as nuclear magnetization) of a hydrogen atom constituting the fat is not T1-recovered, so that a decrease in a fat suppression effect due to an inversion pulse is suppressed.

In addition, a method of removing an influence of a blood flow in addition to the fat suppression has also been developed, for example, JP2018-202122A discloses a method of executing two pulse sequences for data collection with different timings from the IR pulse and obtaining a difference in pulse sequence signal acquisition started after the two pulse sequences for data collection. In addition, Japanese Patent No. 6420583 discloses that a pre-pulse (MSDE) that suppresses a blood flow signal to a low signal is applied after the fat suppression pulse is applied.

### SUMMARY OF THE INVENTION

The IR pulse is applied and the tissue other than the fat is in a state in which the nuclear magnetization thereof remains at the null point at which the nuclear magnetization of the fat is zero, but the contrast of the image obtained from the data collected in that state is strongly affected by the blood increase, and a slight change in a T1 value and a T2 value of the cell is buried in the change in contrast due to the blood increase. In addition, the T1 value and the T2 value are changed depending on the pathological condition such as a tissue accompanied by inflammation and a cancer not accompanied by inflammation, but it is difficult to visualize the T1 value and the T2 value in a discriminable manner for the same reason. On the other hand, in a case in which the blood in the extracellular space is suppressed by using a motion sensitized driven equilibrium (MSDE) method of making the blood flowing in the blood vessel to be a low signal, contrast representing a change in the cells leaked to the inflammation may be obtained.

In the MSDE, since an MSDE pulse group or an MSDE sequence in which the RF pulse group and the diffusion-weighted gradient magnetic field pulse (MPG) are combined is used, there is an effect of weighting the T2 contrast. The T2 contrast is stronger as a time (duration) for executing the MSDE pulse group is longer. Therefore, there is a problem in that it is difficult to obtain desired T1 contrast by adding the MSDE. It is considered that the T2 contrast can be controlled to some extent by increasing the time from the end of the MSDE to the start of the data collection sequence, but in a case in which this time is too long, a blood flow suppression effect of the MSDE is reduced.

In addition, in a tissue in which inflammation has progressed among the inflamed tissues, iron tends to be deposited, and the T2 value is shortened in such a tissue. As a result, there is an object that, even in a case in which the duration of the MSDE is the same, the T2 contrast is too strong, and thus the value is shifted from a desired T2 contrast.

In a case in which the MSDE is added in this way, the adjustment taking into consideration the desired contrast is required, but the fat suppression imaging technique taking into consideration the blood flow suppression in the related art has not yet achieve the adjustment of the MSDE.

An object of the present invention is to provide an MRI apparatus that can suppress fat, minimize an influence on T1 contrast or T2 contrast, and acquire an image in which has excellent discrimination ability between inflamed tissue and normal tissue.

In order to achieve the above-described object, in the present invention, in a case in which imaging using the IR pulse as the fat suppression pulse is executed, a sequence (blood flow suppression sequence) of suppressing the blood flow signal via the MSDE method is added before the data collection sequence, and a length (duration) of the blood flow suppression sequence and a time (hereinafter, also referred to as a delay time) from the end of the blood flow suppression sequence to the start of the data collection sequence are controlled. The control is executed such that desired contrast is estimated based on imaging conditions and imaging parameters of the data collection sequence, and the desired contrast is obtained.

That is, an aspect of the present invention relates to an MRI apparatus comprising: an imaging unit that executes a fat suppression pulse sequence including an IR pulse and a data collection sequence; and a processor including an imaging controller that controls the imaging unit. The imaging controller executes control of causing the imaging unit to execute the fat suppression pulse sequence including a blood flow suppression sequence after the IR pulse is applied and before the data collection sequence is started. The processor further includes a blood flow suppression sequence adjustment unit that adjusts at least one of a duration of the blood flow suppression sequence or a delay time from end of the blood flow suppression sequence to end of application of a preparation pulse.

Another aspect of the present invention relates to a control method of an MRI apparatus including an imaging unit that executes a fat suppression pulse sequence including an IR pulse and a data collection sequence, the control method comprising: executing a blood flow suppression sequence of suppressing signals from moving spins after the IR pulse is applied and before the data collection sequence is started; and controlling at least one of a duration of the blood flow suppression sequence or a delay time from end of the blood flow suppression sequence to start of the data collection sequence, in accordance with contrast of an image obtained by the data collection sequence.

According to the aspects of the present invention, by controlling the duration (T_{MSDS}) of the blood flow suppression sequence and the delay time (TD) from the end of the sequence to the data collection, it is possible to prevent the blood flow from making it difficult to discriminate the difference between the T1 value and the T2 value of the tissue, to prevent the T2 contrast from being too strong, and to obtain an image having the desired contrast.

In addition, according to the aspects of the present invention, since a system can automatically control the duration (T_{MSDS}) of the blood flow suppression sequence and the delay time (TD), which are difficult to adjust via user settings, the optimization of the fat suppression imaging to which the blood flow suppression sequence is added can be achieved without imposing a burden on an operator.

### BRIEF DESCRIPTION OF THE DRAWINGS

FIG. 1 is a diagram showing an overall configuration of an MRI apparatus.

FIG. 2 is a functional block diagram of a computer (processor).

FIG. 3 is a diagram showing a fat suppression pulse sequence using an IR pulse.

FIG. 4 is a diagram showing behavior of spins in the pulse sequence of FIG. 3.

FIG. 5 is a diagram showing an example of a fat suppression pulse sequence including an MSDE sequence adopted in the present embodiment.

FIGS. 6A to 6C are diagrams showing an example of an MSDE pulse group.

FIG. 7 is a diagram showing behavior of spins in the pulse sequence of FIG. 5.

FIG. 8 is a diagram showing a flow of processing according to Embodiment 1.

FIG. 9 is a diagram showing an example of a UI screen for setting imaging conditions.

FIG. 10 is a diagram showing an algorithm for determining an optimal value of the MSDE sequence.

FIG. 11 is a diagram showing an effect according to Embodiment 1.

FIG. 12 is a diagram showing an example of a UI screen that displays diagnosis support information.

FIG. 13 is a diagram showing a fat suppression pulse sequence according to Embodiment 2.

FIG. 14 is a diagram showing an example of k-space data collection according to Embodiment 2.

### DESCRIPTION OF THE PREFERRED EMBODIMENTS

Hereinafter, embodiments of an MRI apparatus and a control method of an MRI apparatus according to an embodiment of the present invention will be described with reference to the accompanying drawings.

First, an overall configuration of the MRI apparatus to which the present invention is applied will be described.

### Configuration of MRI Apparatus

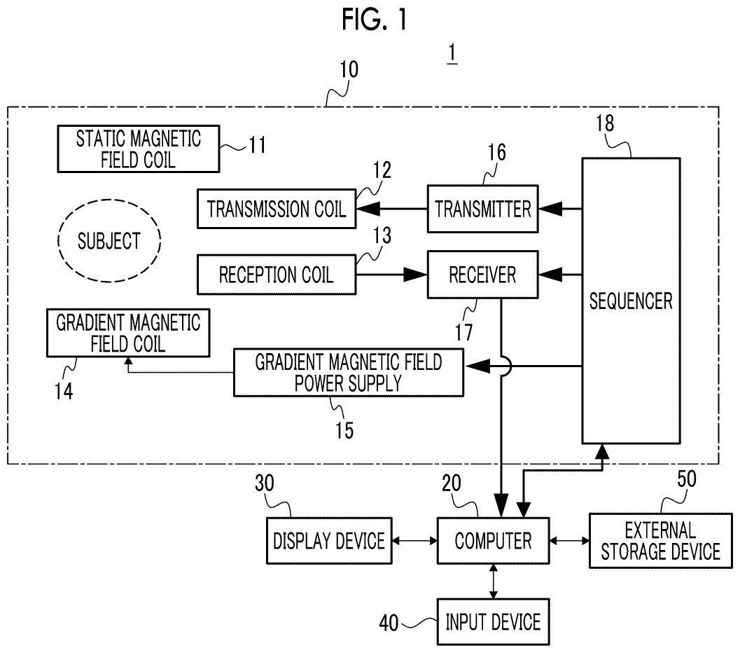

As shown in FIG. 1, an MRI apparatus 1 according to the present embodiment comprises a static magnetic field generator, such as a static magnetic field coil 11, which generates a static magnetic field in a space in which a subject is placed, a high frequency coil for transmission 12 (hereinafter, simply referred to as a transmission coil) and a transmitter 16 that transmits a high frequency magnetic field pulse (RF pulse) to a measurement region of the subject, a high frequency coil for reception 13 (hereinafter, simply referred to as a receive coil) and a receiver 17 that receive a nuclear magnetic resonance signal generated from the subject, a gradient magnetic field coil 14 that applies a magnetic field gradient to the static magnetic field generated by the static magnetic field coil 11, a gradient magnetic field power supply 15 that is a driving power supply of the gradient magnetic field coil 14, a sequencer 18, and a computer 20. The respective units of the MRI apparatus 1 excluding the computer 20 are collectively referred to as an imaging unit 10.

The MRI apparatus 1 includes a vertical magnetic field method and a horizontal magnetic field method depending on a direction of the generated static magnetic field, and the static magnetic field coil 11 is adopted in various forms in accordance with the method. The gradient magnetic field coil 14 consists of a combination of a plurality of coils that generate the gradient magnetic field in three axial directions (x direction, y direction, and z direction) orthogonal to each other, and is driven by the gradient magnetic field power supply 15. By applying the gradient magnetic field, the position information can be added to the nuclear magnetic resonance signal generated from the subject.

It should be noted that, in the example shown in the drawing, a case is shown in which the transmission coil 12 and the receive coil 13 are separate from each other, but one coil that serves as both the transmission coil 12 and the receive coil 13 may be used. The high frequency magnetic field applied by the transmission coil 12 is generated by the transmitter 16. The nuclear magnetic resonance signal detected by the receive coil 13 is transmitted to the computer 20 through the receiver 17.

The sequencer 18 controls the operations of the gradient magnetic field power supply 15, the transmitter 16, and the receiver 17, controls timings of applying the gradient magnetic field and the high frequency magnetic field and receiving the nuclear magnetic resonance signal, and executes the measurement. A time chart of the control is called a pulse sequence, and various pulse sequences different depending on an imaging method are stored in a storage device or the like provided in the computer 20 in advance. In a case in which the imaging method or an imaging part is determined, the sequencer 18 reads out a predetermined pulse sequence, calculates an imaging sequence used for imaging based on imaging conditions set by a user, and controls the imaging unit 10 to collect the nuclear magnetic resonance signal in accordance with the imaging sequence.

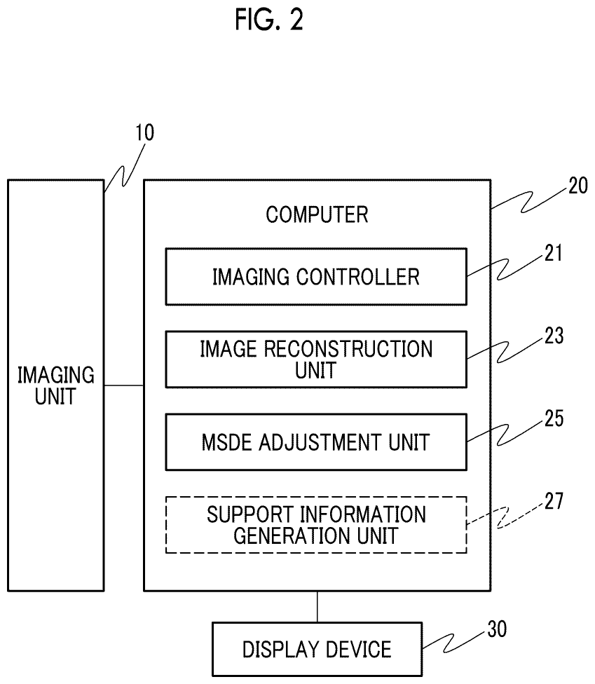

The computer 20 is an information processing apparatus comprising a CPU, a memory, a storage device, and the like, and controls the operations of the respective units of the MRI apparatus via the sequencer 18 and executes various types of operation processing including image reconstruction using measurement data collected by the imaging unit 10. Therefore, as shown in FIG. 2, the computer 20 comprises functional units such as an imaging controller 21 and an image reconstruction unit 23. These functions are executed by reading a program stored in the storage device via the computer 20. However, it is also possible to achieve a part of the functions by a programmable IC such as an ASIC or an FPGA.

Further, the computer 20 according to the present embodiment has a function of adjusting, in a case of executing a data collection sequence to which the IR pulse and the blood flow suppression sequence are added, a duration of the blood flow suppression sequence and a start timing of the data collection sequence (delay time from the blood flow suppression sequence) in accordance with the desired contrast in the data collection sequence. As the blood flow suppression sequence, a known pulse sequence including a large intensity gradient magnetic field pulse (MPG pulse) having an effect of diffusing moving spins can be used, but in the present embodiment, as an example, a case is described in which an MSDE pulse group, which is a typical blood flow suppression sequence having an extracellular diffusion blood flow suppression effect, is used, and in FIG. 2, a sequence adjustment unit that adjusts the blood flow suppression sequence is referred to as an MSDE adjustment unit 25. However, the present invention does not exclude a case in which the blood flow suppression sequence other than the MSDE is used. For example, it is also possible to use an MPG pulse sequence such as a bipolar MPG such as a velocity encoding (VENC) that does not include the RF pulse.

Further, the computer 20 may comprise a support information generation unit 27 having a function of estimating or calculating information for supporting a diagnosis by using an image obtained by a fat suppression pulse sequence. Details of the functions of the computer 20 will be described later.

The computer 20 is connected to a display device 30, an input device 40, an external storage device 50, and the like. The display device 30 is an interface that displays a result obtained by the operation processing or the like to the user (operator). The input device 40 is an interface for the user to input conditions, parameters, and the like required for the measurement or the operation processing executed in the present embodiment. The display device 30 and the input device 40 are collectively referred to as a UI unit. The user can input, for example, the number of echoes to be measured, scan parameters (imaging conditions) such as an echo time TE and an echo interval ITE, via the UI unit. The external storage device 50, together with the storage device in the computer 20, holds data used in various types of operation processing executed by the computer 20, data obtained by the operation processing, input conditions, parameters, and the like.

Based on the above-described configuration, an outline of an operation of the MRI apparatus according to the present embodiment will be described.

In a case of the imaging, an examination part is specified, the imaging method and the imaging conditions are set, and the imaging is started. In a case in which the imaging method or the imaging conditions are set in advance as examination protocols, the examination protocols are read. The imaging method and the imaging conditions are set, and the pulse sequence used for the imaging, TE (echo time), TR (repetitive time), FOV, and the like, which are the scan parameters of the pulse sequence, are determined. In the present embodiment, the fat suppression pulse sequence accompanied by the blood flow suppression is set. Here, as an example, the blood flow suppression sequence is assumed to be the MSDE. The user can further set a type of the MSDE, a b-value, and the like.

In a case in which the fat suppression pulse sequence accompanied by the blood flow suppression is set, the MSDE adjustment unit 25 estimates the contrast intended for the imaging by using a T1 value or a T2 value that is registered in advance for the magnitude of the IR pulse or the tissue included in the examination part, and adjusts the duration of the MSDE applied after the IR pulse and the delay time TD from the MSDE to the start of the data collection sequence. In the adjustment of the duration of the MSDE or the delay time, a range of the duration (minimum duration) or the delay time (minimum delay time) that can be achieved from the imaging conditions set by the user and the conditions (such as the type and the b-value) of the MSDE is determined, and the duration and the delay time in which a ratio between the signal values of the respective tissues included in the imaging part is the desired contrast are determined within the range. The operation executed by the MSDE adjustment unit 25 will be described in detail in the embodiment described later.

After the adjustment via the MSDE adjustment unit 25, the imaging controller 21 executes control of causing the imaging unit 10 to execute the fat suppression pulse sequence under the adjusted conditions. In addition, as necessary, the imaging controller 21 executes the fat suppression pulse sequence including the blood flow suppression sequence and the fat suppression pulse sequence not including the blood flow suppression sequence for the same subject. The support information generation unit 27 generates an image (difference image or ratio image) in which a difference in the influence of the blood flow can be seen or the analyzed data thereof, by using the measurement data obtained by these two fat suppression pulse sequences or the image reconstructed by the image reconstruction unit 23 from the measurement data. The support information generation unit 27 may display the generated diagnosis support information on the display device 30 or may transmit the generated diagnosis support information to a database such as PACS together with image information.

According to the present embodiment, by controlling the duration of the blood flow suppression pulse or the delay time of the data collection and executing the imaging, it is possible to obtain the image in which the influence of the fat signal is suppressed, the signal from the blood in the extracellular space is suppressed, and the desired contrast is obtained in a data collection period.

Hereinafter, before description of a specific embodiment of the function of the MSDE adjustment unit 25, the fat suppression pulse sequence will be described.

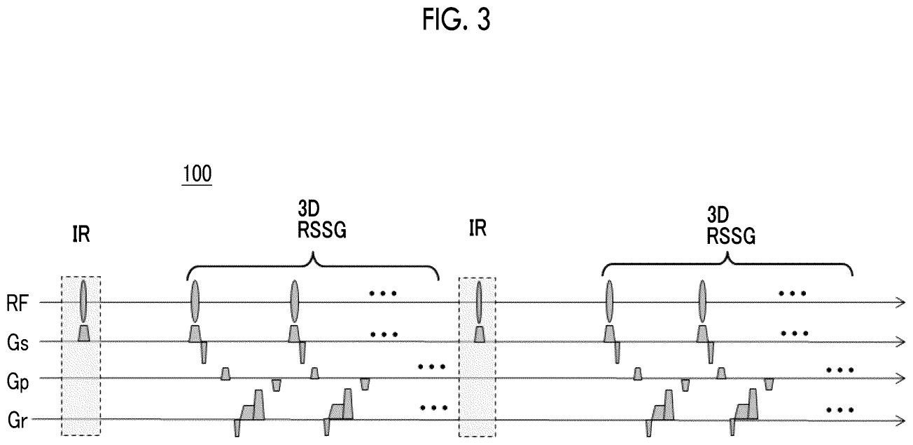

As an example of the fat suppression pulse sequence (excluding the MSDE), a pulse sequence 100 using a RF-spoiled steady state gradient-echo (3D-RSSG) as the data collection sequence is shown in FIG. 3. In FIG. 3, RF indicates an application timing of the RF pulse, and Gs, Gp, and Gr indicate application timings of the gradient magnetic field pulses in a slice direction, a phase encoding direction, and a readout direction, respectively.

The 3D-RSSG is a data collection sequence of repeatedly executing selecting a three-dimensional region (voxel) having a predetermined thickness, applying the RF pulse, applying a phase encoding gradient magnetic field pulse, generating and collect a gradient echo via a readout gradient magnetic field pulse, while changing the intensity of the phase encoding gradient magnetic field pulse, and further repeatedly executing the repetition while changing the intensity of the slice encoding gradient magnetic field pulse to collect three-dimensional k-space data, and there are various methods for the order of the phase encoding and slice encoding, and all of these methods can be adopted.

In this fat suppression pulse sequence 100, first, the IR pulse is applied before the data collection sequence (3D-RSSG), and the data is collected at a point in time (that is, a null point) at which the longitudinal magnetization of the fat disappears or in the vicinity thereof.

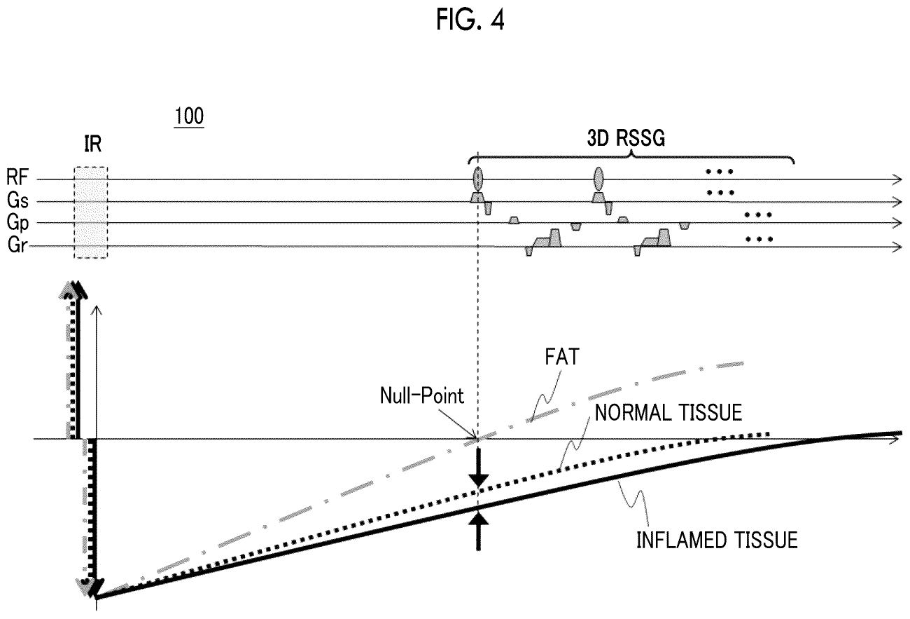

FIG. 4 shows behaviors of spins (longitudinal magnetization) of the fat and spins of other tissues in this case. In the drawing, a horizontal axis represents a time axis, and a vertical axis represents the magnitude of the longitudinal magnetization. As shown in the drawing, in the null point at which the longitudinal magnetization of the fat disappears, the tissue other than the fat, for example, a normal tissue or an inflamed tissue, which has a longer T1 than the fat, has the remaining longitudinal magnetization. By starting the data collection sequence from this point in time, it is possible to obtain the image in which the fat is suppressed. Further, since the T1 values are different between the normal tissue and the inflamed tissue, a contrast difference also occurs in these tissues, and for example, a T1-weighted image with the contrast difference can be obtained. However, for the inflamed tissue, since the blood flow is increased in the vicinity of the cells, the difference in the T1 value or the T2 value between the tissues is buried due to the influence of the blood flow, and it is difficult to obtain a fine contrast of the cells, particularly for the inflamed tissue.

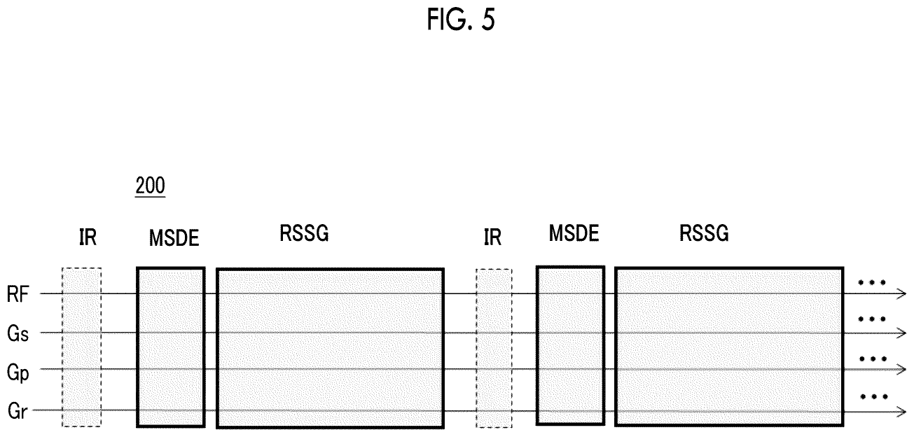

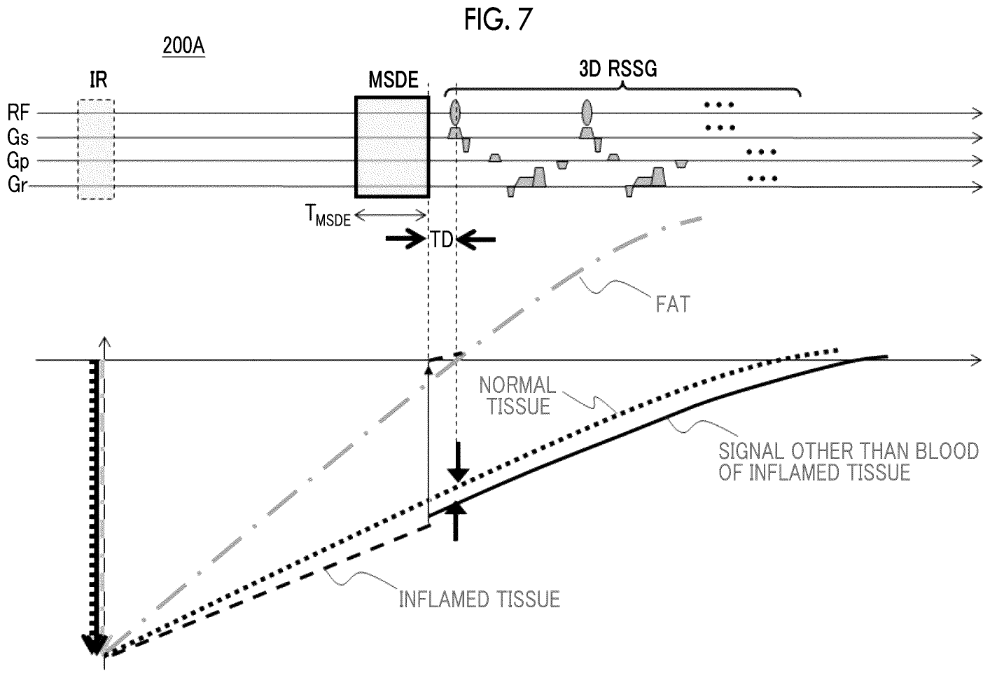

In the imaging sequence according to the present embodiment, a sequence (hereinafter, referred to as an MSDE sequence) consisting of a blood flow suppression pulse group is added immediately before the data collection sequence, thereby suppressing the signal from the blood flow present in the extracellular space. FIG. 5 and FIG. 7 show an example in which the MSDE sequence is added to the fat suppression pulse sequence shown in FIG. 3 (fat suppression pulse sequences 200 and 200A). As shown in the drawing, the MSDE sequence is inserted between the application of the IR pulse in the fat suppression pulse sequence and the null point TI of the fat.

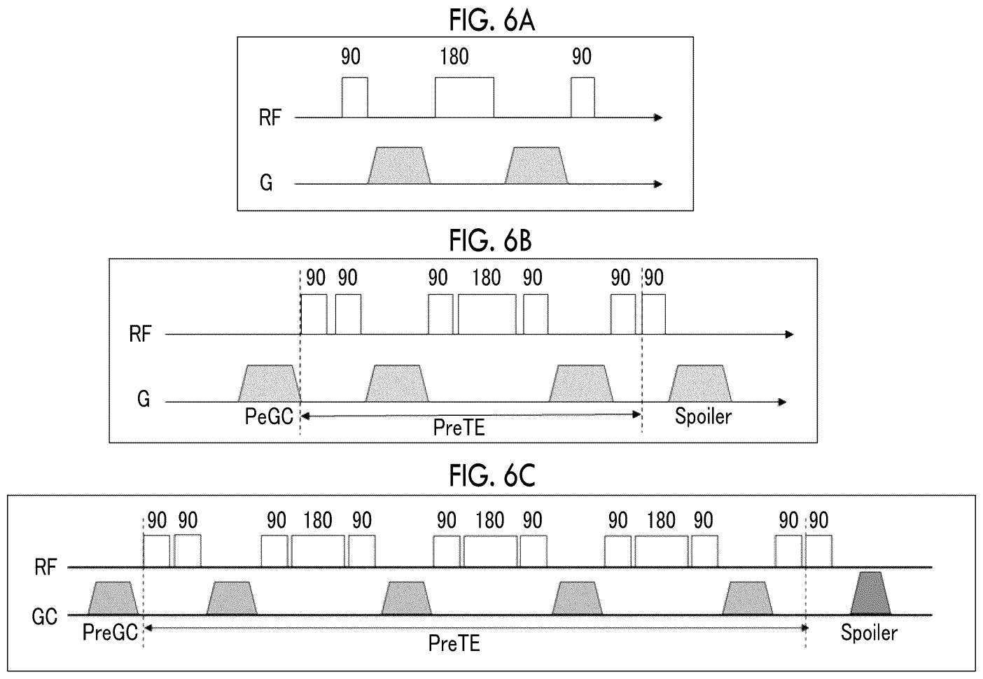

The MSDE sequence consists of a composite pulse (pulse group) in which the RF pulse group and the motion probing gradient (MPG) pulse are combined, and various sequences are known. FIGS. 6A to 6C show an example thereof. FIG. 6A is a most basic sequence in which a 180-degree pulse is disposed between two 90-degree pulses, and the MPG pulse is disposed on both sides of the 180-degree pulse of the RF pulse group. Further, as shown in FIGS. 6B and 6C, there are a sequence in which three groups of the RF pulse groups are used and a sequence in which the RF pulse groups are five groups and a spoiler gradient magnetic field is disposed on both sides of the sequence, and any of these sequences can be adopted. However, the effect of suppressing the blood flow and the contrast of the obtained image are different depending on the number of the RF pulse groups, the pulse interval PreTE, the intensity of the MPG, and the like, and in the present embodiment, the duration of the sequence or the delay time of the data collection sequence executed after the sequence are adjusted in accordance with the desired contrast.

Hereinafter, the significance of the adjustment of the MSDE sequence will be described with reference to the behavior of the longitudinal magnetization shown in FIG. 7.

Although, even in the fat suppression pulse sequence 200A shown in FIG. 7, the change in the longitudinal magnetization of the fat and the normal tissue is the same as in the fat suppression pulse sequence shown in FIG. 3, in the longitudinal magnetization of the inflamed tissue including the longitudinal magnetization of the blood flow, in a case in which the MSDE is added, only the longitudinal magnetization of the blood flow approaches zero, the signal other than the blood remains, and the contrast difference between the longitudinal magnetization of the tissue other than the blood and the normal tissue is clear.

In such an MSDE, as the duration thereof is longer, the blood flow suppression effect is higher, but the signal attenuation occurs in accordance with the transverse relaxation time T2 of the tissue, so that the T2 contrast is weighted. In a case in which the data collection sequence accompanied by the fat suppression is the T1-weighted imaging, the T2 contrast is weighted, and thus the desired T1 contrast cannot be obtained. On the other hand, by increasing the time (delay time) from the MSDE to the start of the data collection sequence, the increase in the T2 contrast due to the MSDE can be suppressed, but in a case in which the delay time is too long, the effect of suppressing the blood flow via the MSDE is reduced. It is extremely difficult for the user to adjust the duration of the MSDE and the delay time each time from the viewpoint of the imaging procedure as well as the technique.

In the present embodiment, the MSDE adjustment unit 25 automatically adjusts the duration T_{MSDE} of the MSDE and the TD in accordance with the contrast of the image to be acquired by the user, thereby enabling the imaging with the desired contrast in which the fat is suppressed and the blood flow is suppressed.

As described above, the duration of the MSDE can be adjusted by the number of the RF pulse groups, the application time of the MPG pulse, and the interval between the pulses indicated by PreTE in FIG. 6.

Hereinafter, an embodiment of specific processing of the adjustment via the MSDE adjustment unit 25 will be described.

### Embodiment 1

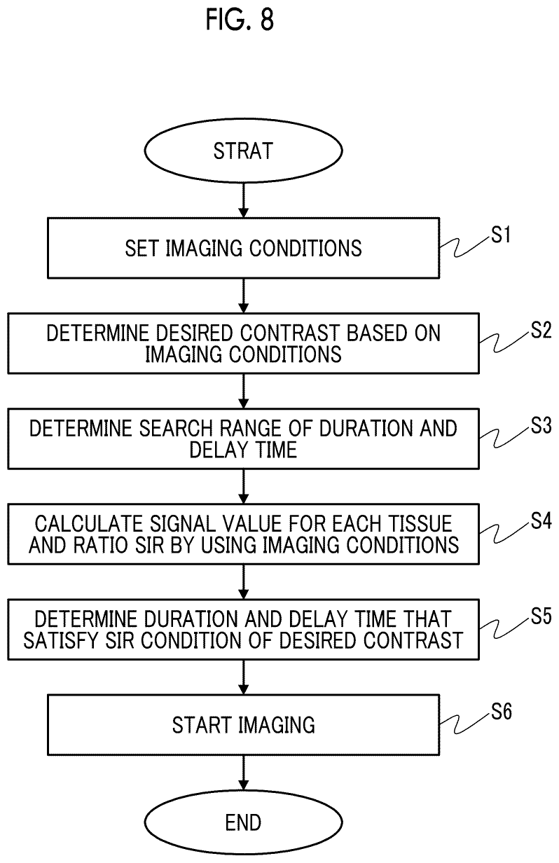

In the present embodiment, as an example, a case will be described in which the data collection sequence is the 3D-RSSG shown in FIG. 7. FIG. 8 shows a flow of processing up to the start of the imaging.

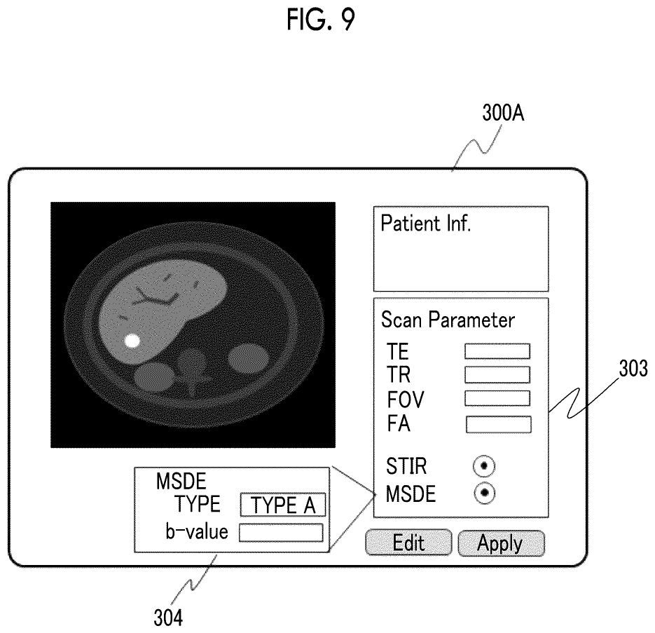

First, the operator sets subject information, the imaging part, and the like, selects the fat suppression pulse sequence including the MSDE sequence as the imaging sequence as shown in FIG. 4, and sets the imaging conditions such as the scan parameters other than the MSDE (S1). The imaging conditions can be set, for example, via a parameter setting block 303 of an UI screen 300A as shown in FIG. 9. The imaging conditions may include information such as whether the imaging is two-dimensional imaging or three-dimensional imaging, and the image type (T1-weighted image, T2-weighted image, PD image), in addition to the scan parameters (TE, TR, FA, TI, and FOV). In addition, in a case in which the MSDE application is set in the setting of the imaging conditions, the operator sets the conditions of the MSDE sequence, such as the type of the MSDE, the b-value which is an indicator of the intensity of the MPG pulse, and the like. For example, the setting is executed by a block 304. The type of the MSDE, the number of the RF pulse groups, and the like may be set to default and standard values, and the user may adjust these values.

The MSDE adjustment unit 25 reads the set imaging conditions, and calculates the temporal conditions of the MSDE, that is, the optimal MSDE sequence length and the delay time TD in accordance with the desired contrast of the operator.

Therefore, first, the MSDE adjustment unit 25 determines the contrast that is the base for the calculation based on the set conditions, that is, whether the contrast is T1-weighted, PD-weighted, or T2-weighted (S2). As a method of the determination, for example, in a case in which the types of these contrast image is included as the set information, the types of the contrast images can be used. In addition, a determination may be made with reference to the TE (echo time) and the TR (repetitive time) set as the scan parameters. In general, in an SE-based sequence, in a case in which the TR is shorter than the T1 value of the tissue, a difference in the T1 value between the respective tissues is reflected in the image, and in a case in which the TE is much shorter than the T2 value, an image in which the influence of the T2 value of each tissue is small is obtained. That is, in a case in which both the TR and the TE are short, the T1-weighted image is obtained. Therefore, in a case in which the TE is compared with a predetermined threshold value and the TE exceeds the threshold value, it is determined as being T2-weighted imaging. In a case in which the TE is equal to or less than the predetermined threshold value, the TR is compared with a predetermined threshold value, and in a case in which the TR is equal to or less than the threshold value, it is determined as being T1-weighted imaging. The result of the determination is used for a determination step of subsequent processing.

Next, the MSDE adjustment unit 25 determines the duration T_{MSDE} of the MSDE and the delay time TD until the start of the data collection sequence based on the desired contrast and the set imaging conditions by the following procedure (S3 to S5).

### S3

First, an achievable minimum duration T_{MSDEmin} of the MSDE and the minimum delay time TDₘᵢₙ are determined. As the minimum duration T_{MSDEmin}, based on the apparatus specifications and the set MSDE type (wave number or the like), the minimum duration T_{MSDEmin} of the MSDE sequence that can achieve the set b-value is calculated. That is, T_{MSDEmin}≤T_{MSDE}.

In addition, since the spoiler pulse is required from after the MSDE to the start of the data collection sequence, the application time of the spoiler is set to the minimum value TDₘᵢₙ of the TD.

### S4

Further, the MSDE adjustment unit 25 determines the T1 value and the T2 value of substances A and B to be imaged from the imaging part set in S1. The T1 value and the T2 value of each substance may be previously stored as a table in the system (or external storage device 50), or may be input by the operator via the UI (input device 40).

Next, the signal values S_{A} and S_{B} expected in a case in which the duration T_{MSDE} of the MSDE and the delay time TD are changed are calculated by Expression (1) by using the T1 value and the T2 value of each substance, and the signal ratio SIR_{A/B} between the substance A and the substance B is calculated by Expression (2).

$${S}_{A ( TD , {T}_{MSDE} , {T}_{1 A} , {T}_{2 A} )} = {M}_{0} ( 1 - exp ( - α \frac{{T}_{MSDE}}{{T}_{2 A}} - \frac{TD}{{T}_{1 A}} ) ) \qquad ( 1 )$$

$${SIR}_{A / B} = {S}_{A} / {S}_{B} \qquad ( 2 )$$

In the expression, S_{A} is a signal value of the substance A, and T_{1A} and T_{2A} are the T1 value and the T2 value of the substance A, respectively. Here, only the signal value of the substance A is shown as a representative example, but the signal value S_{B} of the substance B can be obtained in the same manner by replacing the T1 value with the T1 value (T_{1B}) of the substance B and the T2 value with the T2 value (T_{2B}) of the substance B.

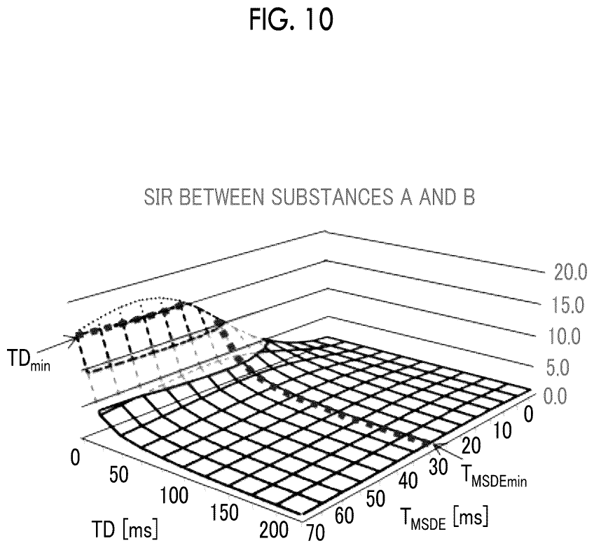

The signal ratio SIR_{A/B} represented by Expression (2) is changed depending on the duration T_{MSDE} of the MSDE and the delay time TD, and for example, as shown in FIG. 10, the signal ratio SIR_{A/B} has a predetermined distribution. FIG. 10 is an example in which T_{1A}=680 ms and T_{2A}=100 ms for the substance A, and T_{1B}=320 ms and T_{2B}=20 ms for the substance B are calculated, and the minimum value TDₘᵢₙ of the TD (=0) and the minimum duration of the MSDE that can be used (T_{MSDEmin} calculated in step S3) are shown by a dotted line, and a range defined by the dotted line is the duration and the delay time that can be achieved for the set MSDE.

### S5

The MSDE adjustment unit 25 determines the optimal point of the map of the signal ratio SIR_{A/B} (FIG. 10) with reference to the signal ratio SIR_{A/B} calculated in this way and the contrast determined in step S2. For example, in a case in which the imaging is the T1-weighted imaging or the PD imaging, a point is used at which the SIR is equal to or less than an allowable value ΔSIRmin of the contrast change in a range of TDₘᵢₙ≤TD and T_{MSDEmin}≤T_{MSDE}, and further TD+T_{MSDE} is minimized. In a case in which there are a plurality of points satisfying the condition, a point is used at which T_{MSDE} is minimized among points at which TD+T_{MSDE} is minimized.

In a case of the T2-weighted imaging, a point is used at which the SIR is maximized in a range of TDₘᵢₙ≤TD and T_{MSDEmin}≤T_{MSDE} and TD+T_{MSDE} is minimized, and in a case in which there are a plurality of the points, a point is used at which the TD is minimized. It should be noted that, although the blood flow suppression pulse is described as the MSDE, the blood flow suppression pulse is not limited to the MSDE. For example, the blood flow signal can be similarly suppressed by applying the bipolar gradient magnetic field such as velocity encoding (VENC). In this case, the SNR is decreased as compared with the MSDE, but the application time of the T_{MSDE} can be shortened.

As a result of the above-described processing, the conditions of the MSDE in which the desired contrast can be maintained is determined. The conditions of the MSDE are determined, and then the imaging is started (S6). Since the processing of the MSDE adjustment unit 25 is system internal processing, the imaging controller automatically starts the imaging with the completion of the above-described step.

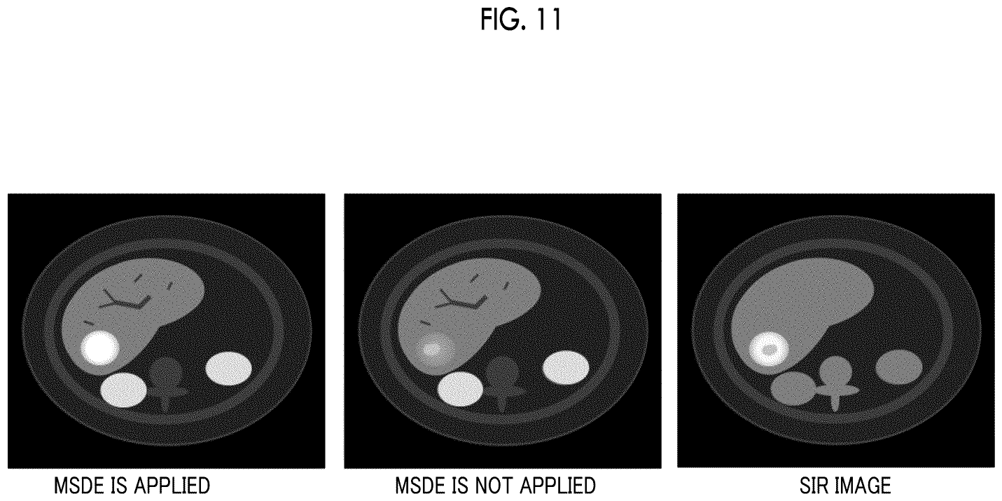

FIG. 11 schematically shows images obtained by the imaging. In FIG. 11, a left side image is an image to which the MSDE is applied, a center image is an image in a case in which the MSDE is not applied, and a right side image is an SIR image (image calculated from MSDE application/MSDE non-application) representing a ratio in a case in which MSDE is applied and a case in which MSDE is not applied. In the image in which the MSDE is not applied, the inflamed tissue in the organ, which is unclear, is visualized with high contrast by applying the optimized MSDE in accordance with the method according to the present embodiment.

As described above, according to the present embodiment, it is possible to prevent the change in the contrast, which is likely to occur due to the addition of the MDSE in the fat suppression imaging, in particular, the shift of the desired contrast, which occurs in a case in which appropriate blood flow suppression is not executed, and it is possible to enhance the visualization ability of the tissue via the blood flow suppression. According to the present embodiment, the temporal condition of the MSDE is automatically adjusted by reading the imaging conditions set by the user, so that the high-quality image can be obtained while reducing the burden on the operator during the examination.

### Application Example

In Embodiment 1, the adjustment of the temporal condition of the MSDE pulse in the fat suppression imaging has been described, but it is also possible to provide useful diagnosis information for an inflammation part such as a joint based on the image. In the present application example, the support information generation unit 27 provides the diagnosis information by using the image to which the MSDE is applied and the image captured without the application of the MSDE. Next, an example of the diagnosis information provided by the support information generation unit 27 will be described.

As shown in FIGS. 4 and 7, in a case in which the MSDE is not applied, a blood signal is mixed in the inflamed tissue, and it is difficult to obtain fine contrast, but in a case in which the MSDE is applied, an image of the inflamed tissue in which the blood signal is suppressed is obtained. Therefore, by taking a difference or a ratio between the image to which the MSDE is applied and the image to which the MSDE is not applied, it is possible to obtain information on how much the blood has accumulated in the extracellular space of the inflamed tissue, that is, the degree of edema of the inflamed tissue. On the other hand, in a case in which the inflammation has progressed and the iron is deposited, the inflamed tissue is not suppressed as the blood, but is visualized with the contrast different between a case in which the MSDE is applied and a case in which the MSDE is not applied, so that the progression of the inflammation can also be estimated from the change in the contrast.

As a premise of the present application example, it is assumed that the imaging controller 21 controls the imaging unit 10 to acquire the image to which the MSDE is not applied, and an MSDE non-application image is obtained. The necessity of the MSDE non-application imaging may be indicated by the operator, for example, via the UI. The support information generation unit 27 calculates a difference or a ratio by using the MSDE image and the MSDE non-application image acquired for the same subject, and generates the difference image or the ratio image (SIR image) as shown on the right side of FIG. 11. Since the MSDE application image maintains the same contrast as the contrast set in the MSDE non-application image, the difference image or the ratio image thereof is an image in which only the effect of the application of the MSDE is reflected.

The image generated by the support information generation unit 27 is displayed on the display device 30. In the difference image or the ratio image, a pixel value of a portion affected by the MSDE, that is, a portion in which the blood is collected, is increased, so that the operator can estimate the degree of inflammation from this image. Further, the support information generation unit 27 can also analyze the degree of inflammation in the ROI by receiving the ROI setting via the operator on the display image. For example, a predetermined threshold value can be set for the pixel value in the ROI, and a value equal to or more than the threshold value and a value equal to or less than the threshold value can be associated with the diagnosis information (degree of edema) related to the blood flow, and the diagnosis information can be presented.

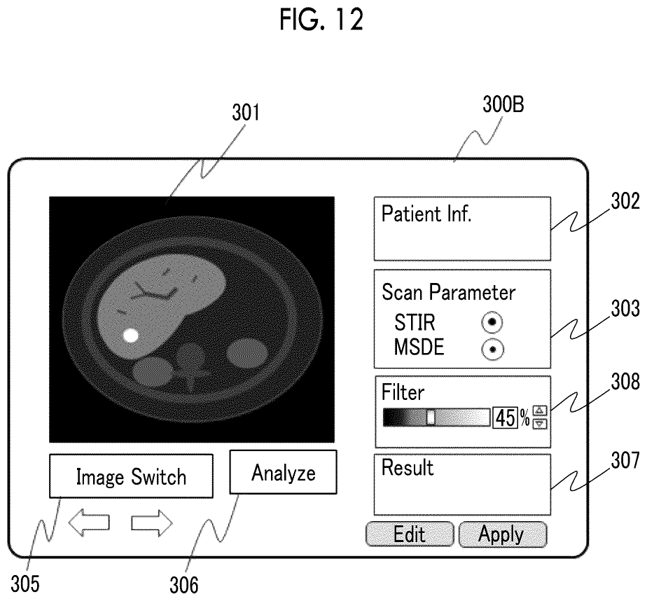

FIG. 12 shows an example of a UI screen 300B used for executing the present application example. In this example, an image display block 301, a subject information display block 302, a scan parameter display block 303, and the like are provided, and the image obtained by the imaging or the image generated by the support information generation unit 27 is displayed in the image display block 301. The display of the image may be switched by operating an image switch button (Image Switch), or the display may be changed to a thumbnail display. In addition, a button (Analyze) 305 for issuing an instruction of the analysis using the difference image or the ratio image may be provided, and the analysis of the image via the support information generation unit 27 may be executed, and the result may be displayed on a result display block 306. In addition, in a case in which the ratio of the region having a low signal intensity is taken, the noise may affect the image reading, so that the influence of the noise may be reduced by removing the noise by performing masking in a case in which the signal value of the original image is equal to or less than the threshold value. The threshold value is set in a filter block 308, and the result is reflected in an image display block 301.

According to the present application example, the operator can confirm the effect of the MSDE and can provide useful information contributing to the diagnosis.

### Embodiment 2

In Embodiment 1, the 3D-RSSG is used as the data collection sequence, but the 2D-RSSG is used in the present embodiment. Also in the present embodiment, the duration T_{MSDE} of the MSDE and the delay time TD are controlled based on the T1 value, the T2 value, and the desired contrast of the tissue, in the same manner as in Embodiment 1. In a case of executing the two-dimensional imaging, it is possible to simply replace the three-dimensional data collection sequence (for example, 3D-RSSG) with a two-dimensional data collection sequence (for example, 2D-RSSG) in the fat suppression pulse sequence 200A shown in FIG. 7, but, in the present embodiment, the imaging efficiency is improved by simultaneously measuring a plurality of slices in one MSDE and by executing the measurement of the slices in a nested manner.

Since the configuration of the apparatus in the present embodiment is the same as the configuration shown in FIGS. 1 and 2, the present embodiment will be described with a focus on the following differences.

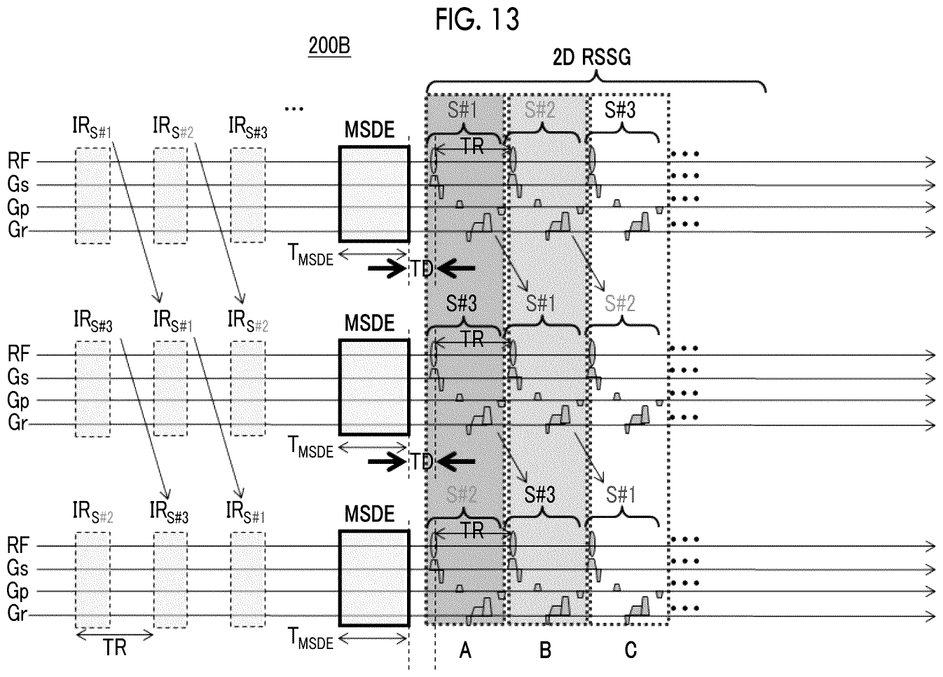

Next, a fat suppression pulse sequence 200B according to the present embodiment will be described with reference to FIG. 13. Here, as an example, a case will be described in which the two-dimensional data collection sequence is the 2D-RSSG. In addition, in order to simplify the description, a case is shown in which the number of slices is three, but the number of slices may be two or three or more. The fat suppression pulse sequence 200B executes a sequence (one shot) from the application of the IR pulse to the data collection sequence at least two times (two shots) or more.

As shown in the drawing, in one shot, a plurality of IR pulses having different slice positions to be selected, IR_{S #1}, IR_{S #2}, and IR_{S #3}, are consecutively applied, and the MSDE sequence is executed before reaching the TI. Since the MSDE has no slice selectivity, the MSDE acts on all spins in all slices in the same manner and suppresses the blood flow signal. After the end of the MSDE sequence, the two-dimensional data collection sequence, that is, the 2D-RSSG is consecutively executed for each of the slices S #1, S #2, and S #3 with the predetermined delay time TD. The duration T_{MSDE} of the MSDE sequence and the delay time TD of the 2D-RSSG are determined by using a method of the calculation based on the first IR pulse and the 2D-RSSG in the step of Embodiment 1 in Embodiment 1.

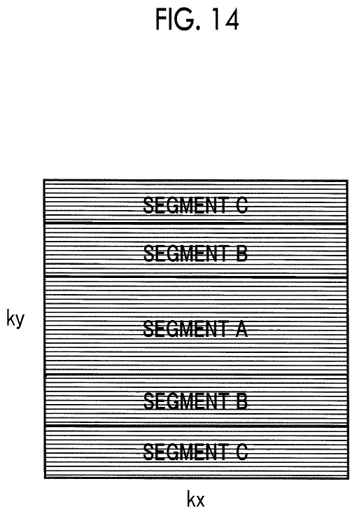

In the 2D-RSSG of each slice consecutively executed within one shot, one or a plurality of echo signals are measured with one TR. In this case, the control is executed such that the k-space data to be collected in each slice is divided into a plurality of segments (same number as the number of slices) having equal areas, and the data of the slices is collected in different segments. In a case in which the number of slices is three, for example, as shown in FIG. 14, the two-dimensional k-space (ky-kz) is divided into a segment A in a low frequency region, a segment B in an intermediate frequency region, and a segment C in a high frequency region. Although FIG. 14 shows an example in which the k-space is divided along the kx axis, the k-space may be divided to have equal areas concentrically from the center of the k-space depending on a sampling method of the k-space (for example, in a case of radial sampling or the like).

As described above, the k-space is divided, and then the slices are looped for each shot to make the contrast constant for each slice by executing the data collection immediately after the MSDE application.

That is, in the first shot (upper part of FIG. 13), the data of the segment A is collected in the slice S #1 that is first executed, the data of the segment B is collected in the slice S #2 that is second executed, and the data of the segment Cis collected in the slice S #3 that is third executed. The data within the same segment may be collected all at once in one TR, or may be collected in a plurality of times.

In the second and subsequent times (middle and lower parts of FIG. 13), the same pulse sequence is executed by changing the order of the slices to be selected. For example, in the second time, the slice selection IR pulses are applied in the order of the slices S #3, S #1, and S #2, and the data collection sequence (2D-RSSG) is executed in this order. Even in this case, in the 2D-RSSG that is executed first, the data of the segment A is collected, the data of the segment B is collected in the second time, and the data of the segment C is collected in the third time. In the third time, the order of the slices is changed such that the slice S #2 is the first, and the data is collected similarly. By executing one rotation of the three shots, the data of all of the segments can be collected uniformly in all of the slices.

As described above, the duration T_{MSDE} of the MSDE and the delay time TD of the 2D-RSSG are set with reference to the first slice, and the diffusion using the MSDE and the T2-weighted degree are different between the second and third slices, but the data in the low frequency region that contributes to the contrast the most is collected in the slice in which the data is first collected in each shot, so that the influence can be reduced by shifting the delay time TD of the data collection sequence of the second and third slices from the set TD.

It should be noted that, in the above description, the data of each segment of the k-space is collected by executing one rotation of three shots, but in a case in which all the k-space data of one slice are collected (all k-space data) in one TR by using EPI or the like as a data collection method, the slice measurement order is looped, and the k-space data collected in each shot is accumulated. In the example of FIG. 13, since the respective pieces of the collection data in a case in which the measurement order is first, second, and third for each slice are integrated, a plurality of pieces of data having different influences on the diffusion and the contrast due to the difference in the TD are averaged, and the unevenness in the image quality between the slices can be prevented.

According to the present embodiment, by executing the measurement of the plurality of slices in a nested manner, the imaging efficiency can be prevented from decreasing due to the application of the IR pulse, which is the slice selection pulse, for each slice. In addition, by dividing the k-space and measuring the data in the high frequency region of the k-space in the data collection sequence of the slice started with the set TD, it is possible to reduce the influence of the difference in the degree of diffusion or the contrast due to the shift of the TD and to ensure the effectiveness of the T_{MSDE} and TD adjustment.

### EXPLANATION OF REFERENCES

1: MRI apparatus
10: imaging unit
20: computer
21: imaging controller
23: image reconstruction unit
25: MSDE adjustment unit
27: support information generation unit

What is claimed is:

1. A magnetic resonance imaging apparatus comprising:
an imaging unit that executes a fat suppression pulse sequence including an IR pulse and a data collection sequence; and
one or more processors that control the imaging unit,
wherein the one or more processors execute control of causing the imaging unit to execute the fat suppression pulse sequence including a blood flow suppression sequence after the IR pulse is applied and before the data collection sequence is started, and
adjust at least one of a duration of the blood flow suppression sequence or a delay time from end of the blood flow suppression sequence to end of application of a preparation pulse.

2. The magnetic resonance imaging apparatus according to claim 1,
wherein the blood flow suppression sequence is an MSDE sequence or a bipolar MPG pulse.

3. The magnetic resonance imaging apparatus according to claim 1,
wherein the blood flow suppression sequence adjustment unit
estimate contrast of an image to be obtained through imaging based on imaging conditions set for the fat suppression pulse sequence, and
determine the duration of the blood flow suppression sequence and the delay time by using the contrast.

4. The magnetic resonance imaging apparatus according to claim 3,
wherein the one or more processors use scan parameters of the data collection sequence and T1 and T2 values of a tissue constituting an imaging target part, as the imaging conditions for estimating the contrast.

5. The magnetic resonance imaging apparatus according to claim 3,
wherein the one or more processors calculate an achievable minimum duration of the blood flow suppression sequence, and determine the duration of the blood flow suppression sequence by using the minimum duration and the estimated contrast.

6. The magnetic resonance imaging apparatus according to claim 5,
wherein the one or more processors calculate the minimum duration of the blood flow suppression sequence by using a set type of the blood flow suppression sequence and a set b-value of an MPG pulse included in the blood flow suppression sequence.

7. The magnetic resonance imaging apparatus according to claim 1,
wherein the data collection sequence is a three-dimensional RSSG sequence.

8. The magnetic resonance imaging apparatus according to claim 1,
wherein the data collection sequence is a two-dimensional data collection sequence of acquiring measurement data for each two-dimensional slice, and
the one or more processors execute control of repeatedly executing, for a plurality of slices, a one-shot sequence of consecutively applying the IR pulse for selecting each slice, executing the blood flow suppression sequence, and then consecutively executing the two-dimensional data collection sequence of each slice after the delay time that is determined, by changing an IR pulse application order for each slice and an execution order of the data collection sequence.

9. The magnetic resonance imaging apparatus according to claim 8,
wherein, in a case of the one-shot sequence is repeatedly executed, the one or more processors cyclically change a region of k-space data to be collected for each slice in accordance with the execution order of the data collection sequence.

10. The magnetic resonance imaging apparatus according to claim 1,
wherein the one or more processors
execute control of causing the imaging unit to execute a first fat suppression pulse sequence including the blood flow suppression sequence and a second fat suppression pulse sequence not including the blood flow suppression sequence, and
further generate diagnosis support information by using measurement data or an image obtained by the first fat suppression pulse sequence and measurement data or an image obtained by the second fat suppression pulse sequence.

11. The magnetic resonance imaging apparatus according to claim 10,
wherein the one or more processors generate a difference image or a ratio image between the image obtained by the first fat suppression pulse sequence and the image obtained by the second fat suppression pulse sequence, as the diagnosis support information.

12. The magnetic resonance imaging apparatus according to claim 11,
wherein the one or more processors estimate presence or absence or a degree of edema of an imaging part by using a ratio or a difference between the measurement data obtained by the first fat suppression pulse sequence and the measurement data obtained by the second fat suppression pulse sequence, as the diagnosis support information.

13. A control method of a magnetic resonance imaging apparatus including an imaging unit that executes a fat suppression pulse sequence including an IR pulse and a data collection sequence, the control method comprising:
executing a blood flow suppression sequence of suppressing signals from moving spins after the IR pulse is applied and before the data collection sequence is started; and
controlling at least one of a duration of the blood flow suppression sequence or a delay time from end of the blood flow suppression sequence to start of the data collection sequence, in accordance with contrast of an image obtained in the data collection sequence.
